(19) United States
(12) Patent Application Publication
(10) Pub. No.: US 2009/0257970 A1
(43) Pub. Date: Oct. 15, 2009

(54) ANTIPERSPIRANT COMPOSITIONS CONTAINING A COPPER SALT AND A PENETRATION ENHANCER AND METHODS OF USING THE SAME

(76) Inventor: Ernest Bloom, Oakland, CA (US)

Correspondence Address: Lawrence F. Rozsnyai, 398 El Paseo, Millbrae, CA, 94030, US

(21) Appl. No.: 12/402,497

(22) Filed: Mar. 12, 2009

Related U.S. Application Data
(60) Provisional application No. 61/069,389, filed on Mar. 14, 2008.

(51) Int. Cl. A61K 8/28; A61K 8/19; A61K 8/20; A61K 8/26; A61Q 15/00

(52) U.S. Cl. 424/66; 424/65; 424/68

(57) ABSTRACT

Antiperspirant compositions including a copper salt and a penetration enhancer are described. The penetration enhancer may be salicylic acid or urea. In addition to a copper salt, the composition may also comprise an aluminum or zirconium salt. Methods of using the compositions are also described. Embodiments may be useful in treating perspiration, hyperhidrosis, and associated undesirable odors of the axillae, hands, feet, lower back, groin, and other areas.

### CROSS-REFERENCE TO RELATED APPLICATION

This application claims priority under 35 U.S.C. § 119 to U.S. Provisional Patent Application 61/069,389 filed Mar. 14, 2008, the entire contents of which are incorporated herein by reference.

### TECHNICAL FIELD

This application relates to antiperspirant compositions, and more particularly to antiperspirant compositions containing a copper salt and a penetration enhancer. Certain compositions further include an aluminum or zirconium salt.

### BACKGROUND

Antiperspirant compositions based on aluminum and zirconium are well known. For example, Shin et al. (U.S. Pat. No. 4,774,079) describe antiperspirant compositions based on aluminum chlorohydrate, aluminum chloride, and an aluminum zirconium polychlorohydrate complex. Parekh et al. (U.S. Pat. No. 6,902,724) describe an aluminum halide antiperspirant with enhanced efficacy. Carrillo et al. (U.S. Pat. No. 6,991,781) describe an enhanced efficacy antiperspirant based on aluminum-zirconium salts. It is generally believed that the mechanism of antiperspirants, such as these, involves the formation of aluminum or zirconium hydroxide precipitates, which plug the sweat ducts and block sweat from reaching the skin surface.

Separately, salicylic acid has been used in certain antiperspirant formulations. For instance, a salicylic acid gel base has been used as a vehicle for aluminum chloride hexahydrate in the treatment of hyperhidrosis, as described in A. Benohanian et al., International Journal of Dermatology, 37, 701-703 (1998), the entire disclosure of which is hereby incorporated by reference. Salicylic acid has also been used in antiperspirant compositions based on aluminum and zirconium (see, e.g., Motley et al., U.S. Pat. No. 5,516,511).

Despite these advances, more effective topical antiperspirant compositions are needed, particularly for those patients suffering from excessive perspiration, and for those patients who do not respond well to available conventional therapies.

### SUMMARY

This disclosure describes antiperspirant compositions including a copper salt and a penetration enhancer. Certain compositions further include a salt of aluminum or zirconium, or a mixture thereof.

Certain implementations may include the following features. The copper salt can be a copper(II) salt. The copper salt can be copper chloride or copper chloride dihydrate. The copper salt may be dissolved in the composition. In some implementations, the copper salt can be included in the composition in an amount from about 0.1% to about 25% by weight of the total volume of the composition. For example, the copper salt may be included in the composition in an amount from about 1% to about 16% by weight, of the total volume of the composition, e.g. about 2%, about 4%, about 8%, or about 15%.

Certain implementations may also include the following features. The penetration enhancer may be included in an amount from about 0.1% to about 50% by weight, of the total volume of the composition. For example, the penetration enhancer may be included in an amount from about 1% to about 20%, e.g. from about 1% to about 10%, e.g. about 4%. The composition may include water. For example, water may be included in an amount from about 0.4% to about 10% by volume, of the total volume of the composition. The composition may include an alcohol, e.g. a straight-chain, branched, or cyclic alcohol having from about 1 to about 8 carbon atoms. The alcohol may be ethyl alcohol. The alcohol may be present in an amount from about 0.1% to about 50% by volume, of the total volume of the solution, e.g. from about 1% to about 40%, e.g. about 20%.

In implementations, the composition may also include an organic carrier. The organic carrier may be selected from the group consisting of straight-chain, branched, and cyclic alcohols, polyols (including diols and glycols), esters, and carbonates having from about 1 to about 6 carbon atoms, and combinations thereof. The organic carrier can be propylene glycol.

In addition to a copper salt and a penetration enhancer, the composition may include an aluminum salt, a zirconium salt, or a mixture thereof. The aluminum or zirconium salt may be a hydrate. For example, the composition may include aluminum trichloride hexahydrate. In certain implementations, the aluminum or zirconium salt may be included in an amount from about 0.1% to about 25% by weight of the total volume of the composition. For example, the aluminum or zirconium salt may be included in an amount from about 1% to about 10%, e.g. about 4%.

Implementations include methods for reducing or preventing perspiration and for treating hyperhidrosis. Implementations also include methods for treating or preventing undesirable odors associated with human sweat glands and skin. Certain methods comprise applying an effective amount of a composition containing a copper salt and a penetration enhancer to the skin of a human subject. In some methods, the composition may also include an aluminum or zirconium salt (or a mixture thereof). Other methods include first applying an effective amount of a composition containing a copper salt and a penetration enhancer followed by applying an effective amount of a composition containing an aluminum or zirconium salt (or a mixture thereof). In some methods, the subject may suffer from social sweating associated with normal daily activities. In composition containing an aluminum or zirconium salt (or a mixture thereof). In some methods, the subject may suffer from exercise-induced sweating.

Certain implementations may have one or more of the following advantages. The compositions can combine in an additive or synergistic manner the effects of copper with the effects of the penetration enhancer. The effects of copper may also be additive or synergistic with the effects of aluminum or zirconium. The compositions may be effective in reducing perspiration and in treating excessive perspiration or hyperhidrosis, particularly with, but not limited to, subjects not responding well to other treatments. The compositions may reduce perspiration for an extended time after application so that application may be necessary only a few times per week, or month, to achieve and maintain the desired degree of reduced perspiration. The compositions may be effective for treating the axillae, hands, feet, lower back, groin, and other areas.

The details of one or more implementations are set forth in the description and examples below. Other features, objects, and advantages will be apparent from the description, examples, and the claims.

### DETAILED DESCRIPTION

This disclosure describes antiperspirant compositions comprising a copper salt and a penetration enhancer.

The term “dissolved” as used herein means that the copper is substantially dissolved in and/or colloidally dispersed in the composition. Concentrations of constituents are by weight of total volume of the composition (weight/volume) for solid constituents such as metal salts, metal salt hydrates, and salicylic acid, and are by volume of the total volume of the composition (volume/volume) for liquid constituents such as water and alcohol. In each case, concentrations are initial concentrations, i.e. those at the time the composition is made (e.g. shortly after the constituent is added to the composition). It is understood, and within the scope of this invention, that concentrations of certain constituents at later times may be different than the initial concentrations (e.g. due to evaporation).

As used herein, the term “copper salt” includes copper ions in the form of or derived from one or more copper salts, including copper salt hydrates. The copper salts used herein are chiefly salts or salt hydrates of copper(II). Examples of copper salts useful herein include, without limitation, chlorides, bromides, iodides, sulfates, nitrates, cyanides, acetates, acetylacetates, oxalates, carbonates, formates, oleates, and maleates, or hydrates thereof, (for example, but not limited to, copper(II) chloride dihydrate (CuCl₂(H₂O)₂) and copper(II) sulfate pentahydrate (Cu(SO₃)(H₂O)₅)). Other inorganic and organic salts may also be used, such as those found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa. (2005) at p. 732, Table 38-5, the contents of which are hereby incorporated by reference herein.

While not wanting to be bound to any particular theory, it is thought that the antiperspirant effect of the copper salts described herein is chiefly due to the therapeutic inhibition of cell metabolic activity by the metal or metal ion. Target cells of this inhibition include cells that are part of, and related to, the sweat glands and associated structures. This proposed mechanism is different from the conventional mechanism of aluminum and zirconium salts. A significant portion of the inhibitory biological action of copper is thought to come from its redox properties. Copper can catalyze the production of very reactive radical ions such as the hydroxyl radical. An increase in free radicals of this type is known as oxidative stress, and is an active area of research in a variety of diseases where copper may play an important role. For instance, oxidative stress can inhibit cell growth or cause cell death, as reported, for example, by L. M. Gaetke and C. K. Chow, Toxicology, 189(1-2), 147-63 (2003); and G. Filomeni et al., J. Biol. Chem., 282(16), 12010-21 (2007). Copper may interfere with other vital cellular functions, including highly energy-dependant membrane pumping functions, which occur in sweat glands. It is therefore thought that copper (e.g. Cu(II) and Cu(I)) may damage, disrupt, or even cause apoptosis or cell death of some the sweat gland cells, thereby reducing or eliminating sweat production.

As used herein, the term “penetration enhancer” means an agent which enables or increases entrance of substances primarily into the lumen of the sweat gland duct. A penetration enhancer may also increase percutaneous absorption through the epidermis, to the dermis, and into the bloodstream. Penetration enhancers of the disclosed compositions include salicylic acid and urea. Other penetration enhancers, such as dimethylsulfoxide (DMSO) may also be used. Other suitable penetration enhancers are listed in Remington (2006), at page 959 (see Table 47-9), which is hereby incorporated by reference in its entirety.

As described herein, salicylic acid and urea may act as penetration enhancers of the active antiperspirant ingredient. Salicylic acid is a keratolytic agent, causing cells of the epidermis to slough off, and causing keratin-lined skin pores and ducts to remain more open. Salicylic acid is known in the treatment of eczema, keratosis pilaris, psoriasis, and related skin conditions. While not wanting to be bound to any particular theory, it is thought that salicylic acid may enhance biological access or absorption of the active antiperspirant ingredient, as disclosed in A. Benohanian et al., above, by improving access to and/or maintaining enhanced opening (patency) of the sweat gland ducts. It is thought that urea may have a similar effect since urea is also a widely used keratolytic agent for treating the skin conditions mentioned above. Salicylic acid may also have other beneficial properties, such as promoting normal skin (non-perspiration related) hydration levels, as also disclosed in A. Benohanian et al., above.

Certain implementations include an organic carrier or vehicle. A suitable organic carrier will have certain desirable properties including the ability to (i) dissolve or disperse the copper salt and the penetration enhancer; (ii) enable a stable, homogeneous composition to be formed; (iii) dry soon after being applied to the skin; and (iv) leave little to no residue after drying. Suitable organic carriers may also be selected for viscosity, lubricity, and other properties. Suitable organic carriers used herein include straight-chain, branched, and cyclic alcohols, polyols (including diols and glycols), esters, and carbonates having from about 1 to about 6 carbon atoms. Examples include, without limitation, methanol, ethanol, n-propanol, isopropanol, n-butanol, 2-methoxyethanol, 2-ethoxyethanol, ethylene glycol, 1,2-propylene glycol, diethylene glycol, isopropanol, isobutanol, diethylene glycol monoethylether, 1,3-butylene glycol, 2,3-butylene glycol, dipropylene glycol, and 2,4-dihydroxy-2-methylpentane. Other suitable organic carriers (including excipients and colloidal systems) may also be used, such as those listed in B. J. Bowman et al., Colloidal Dispersions, Chap. 21, in Remington (2006); and M. M Crowley, Solutions, Emulsions, Suspensions, and Extracts, Chap. 39 in Remington (2006), the disclosures of which are hereby incorporated by reference in their entireties. For clarity, certain organic carriers may also act as penetration enhancers.

In various implementations, water, an alcohol, or both water and an alcohol may be added to the composition to alter the solubility, viscosity, lubricity, and other properties of the composition. Suitable alcohols include straight-chain, branched, and cyclic alcohols having from about 1 to about 8 carbon atoms. Exemplary alcohols include, without limitation, methanol, ethanol, isopropanol, n-propanol, n-butanol, isobutanol, tert-butyl alcohol, 1-hexanol, 1-octanol, and cyclohexanol. Other suitable alcohols may also be used.

Aluminum and zirconium salts may have a synergistic or cumulative antiperspirant effect with copper, since the former metals are thought to work via a different mechanism than copper. Specifically, aluminum and zirconium salts are widely known to form plugs in the sweat glands or otherwise physically block sweat from reaching the surface of the skin, as disclosed, for example, by Kolodzik et al. (U.S. Pat. No. 6,835,373). Therefore, in some implementations, compositions comprising a copper salt and a penetration enhancer also include an aluminum salt, a zirconium salt (which include hydrates thereof) or a mixture thereof. For example, aluminum trichloride hexahydrate (AlCl₃(H₂O)₆) may be used. Other aluminum or zirconium salts known to those skilled in the art may also be used, including halides, oxide-halides, hydroxyhalides such as chlorohydrates, and mixtures thereof. In certain implementations, compositions of a copper salt with an aluminum and/or zirconium salt also include one or more substituents listed above, including an organic carrier, water, and an alcohol.

Methods of using the compositions disclosed herein are also described. In some implementations, methods of reducing or preventing perspiration, of treating hyperhidrosis, and of treating or preventing undesirable odors associated with human sweat glands and skin include applying to the skin of a human an effective amount of a composition comprising a copper salt and a penetration enhancer. In other implementations, a method of reducing or preventing perspiration, of treating hyperhidrosis, or of treating or preventing undesirable odors associated with human sweat glands and skin include first applying a composition comprising a copper salt and a penetration enhancer followed by applying a composition comprising an aluminum or zirconium salt (or a combination thereof). In some implementations, the subject may suffer from social sweating associated with normal daily activities. In some implementations, the subject may suffer from exercise-induced sweating.

The compositions described herein will typically be administered locally and topically at the desired location, for example, but not limited to, the axillae, hands, feet, and groin areas. The compositions may be administered by direct application or with a suitable skin applicator. The composition may also be administered via or as part of any other suitable vehicle, applicator, or deodorant or antiperspirant formulation, including, without limitation, a dropper (e.g. an eye-dropper or pipette), a sponge-top type applicator, a spray, aerosol, roll-on, stick, gel, lotion, cream, salve, or powder.

In accordance with the present invention, there may be employed conventional dermatology, pharmacology, and chemistry techniques within the skill of the art. The invention is further described in the following examples, which do not limit the scope of the claimed invention.

### EXAMPLES

### Example 1

### Preparation of Solutions

To make 1000 ml of solution, a sufficient amount (typically about 400 ml to about 800 ml depending, in part, on the amount of other constituents being added) of propylene glycol was added to a measuring beaker. While stirring at room temperature, the following ingredients were added: 38.4 ml H₂O, 38.4 ml ethanol (anhydrous), 43.3 g salicylic acid, and the following amount of CuCl₂(H₂O)₂:

| Solution | Amount of CuCl₂(H₂O)₂ | Approximate Concentration of |
|---|---|---|
| No. | Added (g) | Copper Salt (as hydrate) (%, w/v) |
| 1 | 20 g | 2 |
| 2 | 40 g | 4 |
| 3 | 100 g | 10 |
| 4 | 150 g | 15 |

Additional propylene glycol was then added until the total volume was 1000 ml. Any undissolved CuCl₂(H₂O)₂ pellets were crushed, and the solution was allowed to stir at room temperature until the copper salt was substantially dissolved (typically for about 12-72 hours or longer). The resulting solutions were transparent and greenish blue in color (in general, the more copper salt present, the deeper the color). The solutions were stored in glass bottles at room temperature.

### Example 2

### Treatment of Axillae

A human female subject was identified as suffering from social sweating associated with various daily activities. She also had underirable axilaary odor(s) in day-to-day social situations. Approximately 1-3 mls of Solution 2 of Example 1 was applied at each treatment to the axillae of the subject, either directly or using an applicator. The subject treated herself at bedtime. Within two successive daily bedtime applications, sweat production and any associated malodor had markedly decreased. Once achieved, this antiperspirant effect could be maintained by similar reapplications approximately once per week.

### Example 3

### Treatment of Feet

A human female subject was identified as suffering from exercise-induced sweating. Approximately 1-3 mls of Solution 3 of Example 1 was applied at each treatment to the feet of the subject, either directly or using an applicator. The subject treated herself at bedtime. Within two successive daily bedtime applications, sweat production markedly decreased. Once achieved, this antiperspirant effect could be maintained by similar reapplications approximately two to three times per week.

### Example 4

### Treatment of Hands

A human male subject was identified as suffering from exercise-induced sweating. Approximately 1-3 mls of Solution 3 of Example 1 was applied at each treatment to the hands of the subject, either directly or using an applicator. The subject treated himself at bedtime. Within two successive daily bedtime applications, sweat production markedly decreased. Once achieved, this antiperspirant effect could be maintained by similar reapplications approximately two to three times per week.

### Example 5

### Treatment of Hands and Feet (Added Strength)

To patients not responding favorably to treatments of Examples 4-5, a stronger solution was used. Approximately 1-3 mls of Solution 4 of Example 1 was applied at each treatment to the hands and feet of two human subjects respectively, either directly or using an applicator. The subjects treated themselves at bedtime. Within two successive daily bedtime applications, sweat production markedly decreased. Once achieved, this antiperspirant effect could be maintained by similar reapplications approximately two to three times per week.

### Example 6

### Treatment of the Lower Back

A human male subject was identified as suffering from exercise-induced sweating. Approximately 1 ml of Solution 2 of Example 1 was applied at each treatment to the lower back of a human male subject, either directly or using an applicator. The subject treated himself nightly. Two or three such applications resulted in approximately 80-90% reduction in sweating, as measured by a comparison of the total amount of sweat absorbed by a cloth. The measurements were made immediately following defined regimens of exercise. The baseline was determined by comparing the data from the pre- and post-treatment periods. This significant antiperspirant effect could be maintained by similar reapplications approximately once or twice per week.

### Example 7

### Synergy with Aluminum

Three human male volunteers aged 54-62 years who had not responded well to commercial over-the-counter aluminum-based antiperspirants. Solutions 5, 6 and 7 were prepared in accordance with Example 1, with the substitution or addition of AlCl₃(H₂O)₆ where indicated.

Solutions 5, 6 and 7 were applied to one axilla of each of the three individuals, sequentially, over consecutive two-week periods. The degree of perspiration was determined from the diameter of sweat rings on cotton shirts worn by the subjects after defined regimens of exercise. The degree of perspiration reduction was determined from the ratio of sweat-ring diameters of the treated vs. non-treated axilla (average values of the three subjects). The results are summarized in Table 1.

TABLE 1

| Solution No. | Ingredients | Reduction in Perspiration |
|---|---|---|
| 5 | AlCl₃(H₂O)₆: 4% | 3% |
| | Salicylic Acid: 4% | |
| | Ethanol: 20% | |
| | Water: 0.4% | |
| 6 | CuCl₂(H₂O)₂: 4% | 22% |
| | Salicylic Acid: 4% | |
| | Ethanol: 20% | |
| | Water: 0.4% | |
| 7 | AlCl₃(H₂O)₆: 4% | 85% |
| | CuCl₂(H₂O)₂: 4% | |
| | Salicylic Acid: 4% | |
| | Ethanol: 20% | |
| | Water: 0.4% | |

Note: Percentage of water is the percentage of water added in addition to water added from the metal salt hydrate.

What is claimed is:

1. An antiperspirant composition comprising:
a copper salt; and
a penetration enhancer.

2. The composition of claim 1 wherein the copper salt is copper chloride.

3. The composition of claim 1 wherein the copper salt is copper(II) chloride dihydrate.

4. The composition of claim 1 wherein the copper salt is included in the composition in an amount from about 0.1% to about 25% by weight, of the total volume of the composition.

5. The composition of claim 4 wherein the copper salt is included in the composition in an amount from about 1% to about 16% by weight, of the total volume of the composition.

6. The composition of claim 1 wherein the penetration enhancer is selected from the group consisting of salicylic acid and urea.

7. The composition of claim 1 wherein the penetration enhancer is included in the composition in an amount from about 0.1% to about 50% by volume, of total volume of the composition.

8. The composition of claim 1, further comprising water.

9. The composition of claim 1, further comprising an alcohol.

10. The composition of claim 1, further comprising an organic carrier.

11. The composition of claim 10 wherein the organic carrier is selected from the group consisting of straight-chain, branched, and cyclic alcohols, polyols, esters, and carbonates having from about 1 to about 6 carbon atoms, and combinations thereof.

12. The composition of claim 11 wherein the organic carrier is propylene glycol.

13. The composition of claim 1, further comprising an aluminum salt, a zirconium salt, or a mixture thereof.

14. The composition of claim 1, further comprising aluminum chloride.

15. An antiperspirant composition comprising:
from about 0.1% to about 25% by weight, of the total volume of the composition, of a copper salt;
from about 0.1% to about 50% by weight, of the total volume of the composition, of a penetration enhancer selected from the group consisting of salicylic acid, urea, and mixtures thereof,
from about 0% to about 10% by volume, of the total volume of the composition, of water;
from about 0% to about 60% by volume, of the total volume of the composition, of an alcohol; and
an organic carrier.

16. The antiperspirant composition of claim 15 further comprising:
from about 0.1% to about 25% by weight of the total volume of the composition of an aluminum salt or a zirconium salt.

17. An antiperspirant composition comprising:
about 4% by weight, of the total volume of the composition, of copper(II) chloride dihydrate;
about 4% by weight, of the total volume of the composition, of salicylic acid;
about 20% by volume, of the total volume of the composition, of ethanol; and polyethylene glycol.

18. The composition of claim 17, further comprising about 4% by weight, of the total volume of the composition, of aluminum chloride hexahydrate.

19. A method of reducing or preventing perspiration comprising applying to the skin of a human an effective amount of the composition of claim 1.

20. A method of treating hyperhidrosis comprising applying to the skin of a human an effective amount of the composition of claim 1.

21. A method of reducing or preventing perspiration or treating hyperhidrosis comprising applying to the skin of a human an effective amount of the composition of claim 13.

22. A method of treating or preventing undesirable odors associated with human sweat glands and skin comprising applying to the skin of a human an effective amount of the composition of claim 1.

23. A method of reducing or preventing perspiration or treating hyperhidrosis comprising:
first applying to the skin of a human an effective amount of the composition of claim 1; and
subsequently applying to approximately the same area an effective amount of a composition containing an aluminum salt, a zirconium salt, or a mixture thereof.

24. An article of manufacture comprising a composition of claim 1.
